(19) United States
(12) Patent Application Publication
(10) Pub. No.: US 2014/0277091 A1
(43) Pub. Date: Sep. 18, 2014

(54) DETACHABLE DELIVERY DEVICE

(71) Applicant: Cook Medical Technologies LLC, Bloomington, IN (US)

(72) Inventor: Mark Breedlove, Carmel, IN (US)

(73) Assignee: Cook Medical Technologies LLC, Bloomington, IN (US)

(21) Appl. No.: 14/205,961

(22) Filed: Mar. 12, 2014

Related U.S. Application Data
(60) Provisional application No. 61/783,235, filed on Mar. 14, 2013.

(51) Int. Cl. A61B 17/12

(52) U.S. Cl. CPC A61B 17/12145
USPC 606/200

(57) ABSTRACT

A system for delivering an implantable medical device is provided including the medical device, a delivery wire that is rotationally coupled to the medical device, and a catheter. The delivery wire includes a spiraling marking extending around the delivery wire to provide visual confirmation that the wire is rotating. The medical device is delivered to a target site within a patient's body, and the delivery wire is rotated to release the medical device from the wire. Observation of the rotating spiral markings helps ensure that the medical device is being released from the wire. The rotational coupling between the delivery wire and the medical device limits the medical device from becoming unintentionally released, and allows for repositioning of the device within the body even after the device has been exposed from the catheter.

### CROSS-REFERENCE TO RELATED APPLICATIONS

This application claims the benefit under 35 U.S.C. §119(e) of U.S. Provisional Application No. 61/783,235, filed Mar. 14, 2013, which is hereby incorporated by reference in its entirety.

### BACKGROUND

A standard procedure used in the treatment of endovascular diseases is the placement of medical devices, such as embolic coils, stents, and dilation balloons, among others, at a desired or targeted site (e.g., aneurysm, etc.) within a patient. The delivery of such a medical device has typically been accomplished by a variety of means, including the use of a catheter along with a pushing wire or a means of injection, as well as a system to which the device is attached during delivery and detached once the device has reached the intended site. These medical devices usually have a contracted shape that allows them to pass through the lumen of the body vessel and an expanded shape that occurs after being deployed at the targeted site.

One specific example, of such a medical device is an embolic or occlusive device that is placed within a body vessel or vasculature of the human body to filter the flow of blood through a vessel in the vasculature or to block the flow of blood within a defect in the vessel, such as an aneurysm. One example among many widely accepted types of occlusive devices is a detachable helical wire coil whose coil windings are sized to engage the wall of the vessel. Detachable coils are usually selected when the anatomy is especially distal and tortuous, a risk of coil displacement exists, or a very precise placement of the coil is required.

### SUMMARY

A system for delivering a medical device to patient's body is provided, the system comprising: an elongate delivery wire having proximal and distal ends; an implantable medical device having proximal and distal ends, the proximal end of the medical device releasably attached to the distal end of the delivery wire; a rotatable connection releasably coupling the proximal end of the medical device to the distal end of the delivery wire, the rotatable connection being disengaged via rotation of the delivery wire relative to the medical device; and a spiraling marking disposed along at least a portion of the delivery wire for indicating rotation of the wire.

In another form, a method for delivering an implantable medical device into a patient's body is provided, the method comprising: inserting a catheter into a patient's body and advancing the catheter toward a target site within the body; inserting a delivery wire and an implantable medical device through the catheter toward the target site, wherein the medical device is releasably attached to the delivery wire via a rotatable connection and the delivery wire includes a spiraling marking disposed thereon; rotating the delivery wire relative to the medical device; visually observing the spiraling marking rotate along with the delivery wire; and in response to rotating the delivery wire, releasing the medical device from its attachment to the delivery wire.

Further areas of applicability will become apparent from the description provided herein. It should be understood that the description and specific examples are intended for purposes of illustration only and are not intended to limit the scope of the present disclosure.

### BRIEF DESCRIPTION OF THE DRAWINGS

The drawings described herein are for illustration purposes only and are not intended to limit the scope of the present disclosure in any way. One skilled-in-the-art will understand that a helical coil is used in the Figures as an example of a medical device and that other medical devices may be utilized without exceeding the scope of the disclosure.

FIG. 1 is a schematic view of a medical device delivery system having a delivery wire and an implantable medical device;

FIG. 2 is a schematic view of one embodiment of a rotatable connection between the delivery wire and the implantable medical device shown disconnected;

FIG. 3 is a schematic view of another embodiment of a rotatable connection between the delivery wire and the implantable medical device shown connected;

FIG. 4 is a side view of the delivery wire illustrating a spiraling pattern thereon;

FIG. 5 is a side view of the delivery wire illustrating another spiraling pattern;

FIG. 6 is a side view of the delivery wire illustrating another spiraling pattern;

FIG. 7 is a side view of the delivery wire illustrating another spiraling pattern;

FIG. 8 is a side view of the delivery wire illustrating another spiraling pattern;

FIG. 9 is a side view of the delivery wire illustrating another spiraling pattern;

FIG. 10 is a side view of the delivery wire illustrating a sleeve with a spiraling pattern thereon;

FIG. 11 is a schematic view of a guidewire and a catheter inserted into a patient's body; and

FIG. 12 is a schematic view of the delivery wire and implantable medical device inserted through the catheter toward a target site within the patient's body.

### DETAILED DESCRIPTION

The following description is merely exemplary in nature and is in no way intended to limit the present disclosure or its application or uses. It should be understood that throughout the description and drawings, corresponding reference numerals indicate like or corresponding parts and features.

Referring to FIGS. 1-12, a medical device delivery system 10 includes a delivery wire 12, a detachable medical device or embolization coil 14 attached to the delivery wire 12, a catheter 16, and a guidewire 18. The delivery wire 12 and coil 14 combine to form a medical device assembly 19 that can be inserted through the catheter 14 for delivery at a target site within a patient's body. Once delivered to the target site, the coil 14 can be detached from the delivery wire 12 for implantation in the desired portion of the patient's body.

With reference now to FIG. 1, the delivery wire 12 can include a proximal portion 20 and a distal portion 22. The proximal portion 20 is intended for manipulation by the user to push the delivery wire 12 and coil 14 through the catheter 16 toward the target site. The distal portion 22 is releasably attached to the coil 14.

The wire 12 can be a solid core wire made from Nitinol or stainless steel. The wire 12 can have a diameter in the range of 0.010 to 0.035 in one form; however, other diameters of the wire 12 could also be used to suit the needs of the user. The length of the wire 12 depends on the location of the target site within the body and the distance that the wire 12 must travel through a tortuous body lumen to reach the target site. It will be appreciated that the length of the wire 12 can be selected from any length known in the art that would be suitable for such a delivery. The wire 12 is capable of being pushed through the catheter 16 to deliver the attached coil 14, as well as having torqueability to enable the wire to be twisted, as further described below. In addition to the solid core construction, the wire 12 could also be in the form of a coiled wire, a braided wire, or some other composite wire that has sufficient pushability and torqueability, as known in the art.

The medical device or embolization coil 14 has a proximal portion 24 and a distal portion 26. The proximal portion 24 is releasably attached to the distal portion 22 of the delivery wire 12. When the coil 14 has been delivered to the target site, the coil 14 can be released from the delivery wire 12 for implantation in the patient's body.

The coil 14 can be any type of embolization coil suited for delivery through an elongate catheter. The coil 14 can be made from platinum, Nitinol, or other known embolization coil materials. The coil 14 can be formed as a solid core wire that is wrapped helically to create an elongate coiled form, and can be further formed or bent into a desired embolization coil shape known in the art. One type of embolization coil shape is a conical shape, while another type of coil 14 is in the form of a flexible coil that can fold over itself to fill the shape of the target site. Another type of coil 14 is in the form of a larger spiral coil. It will be appreciated that other known types of the embolization coil 14 could also be used.

The medical device has been described as the embolization coil 14 above; however other types of implantable or otherwise deliverable medical devices could also be attached to the delivery wire 12 for releasable delivery to the target site. For example, occlusion devices, filter devices, or the like could be attached to the delivery wire 12 for delivery through the catheter 16 and released from the wire 12 after being delivered to the target site. However, for purposes of discussion, the medical device 14 will be referred to as the coil 14.

As described above, the coil 14 is releasably attached to the delivery wire 12. More specifically, the proximal end 24 of the coil 14 is attached to the distal end 22 of the wire 12. Even more specifically, the coil 14 is attached to the wire 12 via a rotatable connection 30. The rotatable connection 30 includes a wire portion 32 and a coil portion 34. The wire portion 32 is mounted to the distal end of the distal portion 22 of the wire 12. The coil portion 34 is mounted to the proximal end 26 of the coil 14. The wire portion 32 is rotatably attached to the coil portion 34, so that rotation of the wire portion 32 will release the coil portion 34 and the coil 14 from the delivery wire 12.

In one form, as shown in FIG. 2, the rotatable connection 30 can be in the form of a screw-type connection 31, where the wire portion 32 can be in the form of a threaded portion 40 at the distal end of the wire 12. The threaded portion 40 can include external spiraling threads 42 to create the screw-type connection 31. The coil portion 34 in this form can be in the form of a collar portion 44 having internal threads 46 that correspond to the threads 42 of the wire portion 32. The wire portion 32 can be screwed into the coil 34 so that the threads 42 engage the threads 46 to create the rotatable connection 30. To release the coil 14 from the wire 12, the threaded portion 40 can be rotated relative to the collar portion 44 in the direction opposite the rotational direction used to connect the two portions. To achieve this relative rotation, the wire 12 can be rotated while the coil 14 remains fixed, or the coil 14 can be rotated while the wire 12 remains fixed. Alternatively, both the wire 12 and the coil 14 can be rotated in opposite directions. However, given the delivery of the coil 14 into the patient's body to the target site and the implantable nature of the coil 14, the coil 14 will generally engage the body vessel and be limiting from rotating, so that in practice the wire 12 will likely be rotated while the coil 14 remains relatively rotationally stationary. The portions 32 and 34 can be integrally formed with the wire 12 and coil 14, respectively, or be separate components attached or mounted thereto.

The rotatable connection 30 can help ensure that the coil 14 remains connected prior to reaching the desired delivery location. It also helps ensure that that the coil 14 can be adjusted once it has been delivered but prior to being released from the wire 12. For example, if the coil 14 is placed too far down the body vessel, the coil 14 can be pulled back. If the coil 14 is not far enough, it can be further pushed. In other systems, a coil can be delivered by being pushed through a catheter by a pusher wire, but without control over the coil once it has been pushed out of the catheter. Moreover, if the size of the coil was incorrect for the body vessel, such as being too small, the user may lose control of the coil and it could migrate down the body vessel to another area within the body, which is undesirable. Solutions for such a problem included the use of another tube or sleeve that housed the coil, but this added undesirable complexity and thickness to the design, while still limiting the ability to reposition the coil after being exposed from the tube in the body vessel.

In another form, as shown in FIG. 3, the rotatable connection 30 can be in the form of an interlocking spiral connection 50. The spiral connection 50 includes a wire portion 52 and a coil portion 54. The wire portion 52 is mounted or attached to the distal end 22 of the wire 12. The coil portion 54 is mounted or attached to the proximal end 24 of the coil 14. The wire portion 52 and coil portion 54 each have corresponding spiraling shapes, so that they can be twisted together to create the spiral connection 50. To release the coil 14 from the wire 12, the coil 14 and wire 12 can be rotated relative to each other such that the wire portion 52 and coil portion 54 rotate relative to each other in a spiraling manner. Rotation in a first direction will increase the connection 50, while rotating in the opposite direction will decrease the connection and ultimately release the coil portion 54 and the coil 14 from the wire 12. The wire portion 52 and coil portion 54 are shown in FIG. 3 as having space between their coils, but the space between the coils could be reduced such that it is a tighter fit.

Of course, in addition to the screw-type connection 31 and the spiral connection 50, other rotatable connections 30 could also be used that utilize relative rotation between components to release them from each other, while preserving the connection absent sufficient rotation.

In practice, the wire 12 is the primary rotated component to create the relative rotation between the wire 12 and the coil 14 to release the coil 14 from the wire 12. As mentioned above, when the coil 14 is delivered from the catheter 16, the coil 14 can contact the body vessel and become anchored, as intended. Thus, the coil 14 is generally fixed rotationally, so the wire 12 can be rotated to release the coil 14 from the wire 12. Additionally, given the placement of the coil 14 within the body, rotating the coil 14 while keeping the wire 12 relatively fixed rotationally can be difficult without the use of additional tools inserted into the body. Thus, the wire 12 is preferably rotated to release the coil 14 therefrom.

With the coil 14 within the body, visualizing the rotatable connection 30 between the wire 12 and coil 14 can be limited relative to direct visualization. It is desirable to ensure that the coil 14 has been released from the wire 12 in response to the rotation of the wire 12, but because the coil 14 is generally not available for direct visualization, it is desirable to observe the rotation of the wire 12. However, determining whether the wire 12 is rotating can be difficult due to low light conditions that can exist in operating rooms or the need to monitor other portions of the procedure, such as the placement of the coil 14. Furthermore, the users of the system 10 are often wearing gloves that can affect the tactile response of the wire 12. Additionally, with the coil 14 generally fixed within the body vessel and the rotatable connection 30 keeping the coil 14 coupled to the wire 12, attempted rotation of the wire 12 could be limited by the rotatable connection 30 being slightly stuck, or tension created in the connection 30 to prevent undesired release could act against initial attempts to rotate the wire 12. Attempts to rotate the wire 12 could also result in the wire 12 slipping within the hands or fingers or the user that is attempting to rotate the wire 12, so what may feel like rotating may not be.

With reference to FIGS. 1 and 4-10, to assist in confirming that the wire 12 is rotating, the wire 12 can include spiraling or helical markings 60 disposed on the wire 12. The markings 60 can be in the form of a first line 62, shown in FIG. 4, spiraling around the wire 12 at a constant rate. Rotation of the wire 12 will create a visual effect of the first line 62 moving along the length of the wire 12, thereby indicating that the wire 12 is being rotated, and indicating that the rotatable connection 30 is being disengaged to release the coil 14.

In another form, shown in FIG. 5, the helical marking pattern 60 can include a second line 64 that spirals alongside the first line 62. The second line 64 can be made of the same color as the first line, or it can be made from a different color to increase contrast and provide additional visualization. In FIG. 5, the second line 64 is shown having a different hatching pattern than the first line 62 to illustrate the different color or appearance, but this hatching is merely illustrative and not necessarily indicative of the appearance of the lines 62 and 64.

The first line 62 can have a first thickness that can be selected from a wide range of line thicknesses to provide the desired visualization effect. The second line 64 can be of the same thickness as the first line 62 (FIG. 5), or the second line 64 can have a thickness that is greater, as shown in FIG. 6, or smaller than the first line 62 to provide another type of contrast of thick and thin lines moving together.

The wire 12 can also include a non-marked portion 66, shown in FIGS. 4-9, that provides contrast to the first line 62 and/or second line 64. The shape of the non-marked portion 66 depends on the size and shape of the first line 62 and/or second line 64. In one form, the first line 62 can spiral around the wire 12 such that distance between adjacent spirals of the line 62 is approximately the same as the thickness of the line 62, to create the appearance of two directly adjacent lines spiraling together, as shown in FIG. 7. Of course, the distance between adjacent spirals of the line 62 can be larger or smaller to change the relative thickness of the non-marked portion 66, as shown in FIGS. 4-6.

The above description of the distance between adjacent spirals of the line 62 can apply to the distance between the first line 62 and the second line 64. In another form, the first line 62 and second line 64 can be arranged so that they spiral together as a pair being a first distance apart, but the spiraling shape is such that the distance between adjacent spirals of the pair is greater than the distance between the lines 62 and 64 that define the pair, as shown in FIG. 8.

In another form, as shown in FIG. 9, the first line 62 can have a spiraling pattern where the distance between adjacent spirals changes as the line 62 spirals about the wire 12. For example, the spacing between adjacent spirals can decrease as the line 62 spirals in the proximal direction. Conversely, the spacing between adjacent spirals can increase as the line spirals in the proximal direction. In another form, the spacing between adjacent spirals of the line 62 can increase and then decrease as the line 62 spirals in the proximal direction. It will be appreciated that other varying distance configurations at different longitudinal points along the length of the wire 12 can also be used.

While the above description has referred to the first line 62 and/or the second line, it will be appreciated that above variations of the line size, shape, color, etc. can apply to both the first line 62 and second line 64. Moreover, it will be appreciated that more than two lines can be used to suit the needs of the user.

The markings 60 can be applied to the wire 12 either directly or indirectly. In one form, the markings 60 can be in the form of Teflon coating that is extruded or painted directly onto the wire 12. In another form, the markings 60 can be applied to a polymer jacket 70 through which the wire 12 is inserted, with the jacket 70 being attached directly to the wire 12, as shown in FIG. 10. It will be appreciated that other manners of applying the spiraling marking 60 can also be used.

In another form, in the case of a coiled or braided type of wire, the markings 60 can be in the form of individual strands or wires that define the overall wire 12. A portion of the strands that form the braided wire 12 can be one color while the other portions are other colors. Adjacent coils that form the coiled wire 12 could be different colors.

The above described markings 60 can be applied to the full length of the wire 12, or the markings can be applied to proximal portion 20 while the distal portion 22 remains free from the markings 60, as shown in FIG. 1. Because the markings 60 are used to determine whether the wire 12 is rotating, the distal portion 22, which is generally disposed inside the body when the wire 12 is rotated, can be free from the markings 60.

The system 10 can further include a torque device 80 (FIG. 1) that can be attached to the proximal portion of the wire 12 to assist the user in rotating the wire 12. The torque device 80 can be clamped to the proximal portion of the wire 12 to provide the user with a more robust portion to grip and rotate the wire 12. However, the wire 12 can still be rotated without the torque device 80.

The wire 12 and coil 14 can each include radiopaque markers 85 (FIG. 1) disposed therein to assist in determining the location of the wire 12 and coil 14 within the body via fluoroscopy. With a fluoroscopic agent introduced into the body lumen, the user can monitor the position of the wire 12 and coil 14 to determine whether the coil 14 has been delivered to the desired target site, and that the coil 14 remains in place after being disconnected from the wire 12. These markers 85 can also help assist in determining that the wire 12 and coil 14 have been disengaged from each other by monitoring the relative position of the markers 85 for the wire 12 relative to the markers 85 for the coil 14. The markers 85 can be disposed at different locations on the wire 12 and coil 14 to suit the needs of the user. In one form, the markers 85 can be attached to each portion of the rotatable connection 30 to help determine whether the connection 30 has been disengaged. The markers 85 can also be applied to the distal end of the coil 14 to determine the depth that the coil 14 has been inserted relative to the vasculature. The markers 85 can be applied to the guidewire 16 and catheter 18 to similarly help determine their location within the vasculature.

Having described the structure of the system 10, the use of the system will now be described.

The system 10 can be inserted into the patient's body using known methods of insertion, such as a percutaneous method. A fluoroscopic agent can be introduced into the vasculature to aid fluoroscopic monitoring of the system 10 within the body. The guidewire 16 can be inserted into the patient's vasculature and advanced toward the target site where delivery of the coil 14 is desired. The position of the guidewire 16 can be monitored using the markers 85.

With reference to FIG. 11, the catheter 18 can be inserted into the body over the guidewire 16 and advanced along the guidewire 16 toward the target site. The catheter 18 positioning can be monitored via fluoroscopy and markers 85 attached to the catheter 18. With the catheter 18 positioned at the target site, the guidewire 16 can be retracted out of the body. Additional contrasting agents can be delivered through the catheter 18 and toward the target site.

With the catheter 18 delivered and the contrasting agent delivered to the target site, the user can determine the size of coil 14 that the user would like to deliver depending on the measured size of the target area. In one form, the wire 12 and coil 14 are in an assembled form, so the user can select the appropriate assembly of the wire 12 and coil 14 depending on the desired size.

If the coil 12 and wire 14 are not pre-assembled, the coil 14 can be attached to the wire 12 via the rotatable connection 30 by rotating the components relative to each other in a first direction to engage the rotatable connection 30.

With reference to FIG. 12, the coil 12 and wire 14 can be inserted through the catheter 18 toward the target site. The position of the coil 14 relative to the target site can be monitored using fluoroscopy. The coil 14 can be advanced out of the catheter 18 by continued advancement, and the deployment of the coil 14 can be monitored. If the position of the coil 14 is undesirable, the user can retract the wire 12 to retract the coil 14, even after the coil 14 has been exposed fully from the catheter 18, because the rotatable connection 30 keeps the wire 12 and coil 14 attached to each other. The coil 14 can then be repositioned into the desired location. This can be repeated until the position of the coil 14 is in the preferred location.

With the coil 14 in the desired location, the coil 14 can be detached from the wire 12. As described above, the wire 12 can be rotated, which rotates the spiral markings 60 thereon. The user can observe the spiral markings 60 to confirm that the wire 12 is rotating. By confirming that the wire 12 is rotating, the user can ensure that the rotatable connection 30 is being disengaged. With rotation of the wire 12 confirmed by observing the rotating spiral markings 60, the user can direct its attention to the coil 14 to watch the coil 14 detach under fluoroscopy. If the user attempts to rotate the wire 12 but the spiral markings 60 are not observed rotating, then the user can determine that the wire 12 has been limited or prevented from rotating, and the user can attempt to determine the cause of the limited rotational ability.

With detachment of the coil 14 confirmed, the wire 12 can be retracted from the catheter 18, and the catheter 18 can be refracted, and the percutaneous access site can be closed to complete the procedure.

The foregoing description of various embodiments of the invention has been presented for purposes of illustration and description. It is not intended to be exhaustive or to limit the invention to the precise embodiments disclosed. Numerous modifications or variations are possible in light of the above teachings. The embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

As a person skilled in the art will readily appreciate, the above description is meant as an illustration of implementation of the principles this invention. This description is not intended to limit the scope or application of this invention in that the invention is susceptible to modification, variation, and change, without departing from the spirit of this invention, as defined in the following claims.

What is claimed is:

1. A system for delivering a medical device to patient's body, the system comprising:
an elongate delivery wire having proximal and distal ends;
an implantable medical device having proximal and distal ends, the proximal end of the medical device releasably attached to the distal end of the delivery wire;
a rotatable connection releasably coupling the proximal end of the medical device to the distal end of the delivery wire, the rotatable connection being disengaged via rotation of the delivery wire relative to the medical device; and
a spiraling marking disposed along at least a portion of the delivery wire for indicating rotation of the wire.

2. The system of claim 1, wherein the medical device comprises an embolization coil.

3. The system of claim 1, wherein the rotatable connection includes a wire portion and a device portion, the wire portion is coupled to the delivery wire, and the device portion is coupled to the device.

4. The system of claim 3, wherein the wire portion includes external threading, the device portion includes a collar having internal threading, and the wire portion is threaded into the device portion.

5. The system of claim 3, wherein the wire portion comprises a first spiraling portion, the device portion comprises a second spiraling portion, and the wire portion and device portion are engaged with each other.

6. The system of claim 3, wherein the wire portion is integrally formed with the delivery wire.

7. The system of claim 1, wherein the spiraling marking comprises a spiraling line extending around the wire.

8. The system of claim 7, wherein the spiraling line comprises a first spiraling line and the spiraling marking further comprises a second spiraling line that has a different color than the first spiraling line.

9. The system of claim 7, wherein the distance between adjacent portions of the spiraling line is generally consistent.

10. The system of claim 7, wherein the distance between adjacent portions of the spiraling line increases in the proximal direction.

11. The system of claim 7, wherein the distance between adjacent portions of the spiraling line decreases in the proximal direction.

12. The system of claim 1, wherein the spiraling portion is in the form of Teflon coating.

13. The system of claim 1, wherein the spiraling portion is painted on the delivery wire.

14. The system of claim 1, wherein the delivery wire includes a polymer jacket surrounding the delivery wire and the spiraling portion is disposed on the polymer jacket.

15. A method for delivering an implantable medical device into a patient's body, the method comprising:
inserting a catheter into a patient's body and advancing the catheter toward a target site within the body;
inserting a delivery wire and an implantable medical device through the catheter toward the target site, wherein the medical device is releasably attached to the delivery wire via a rotatable connection and the delivery wire includes a spiraling marking disposed thereon;
rotating the delivery wire relative to the medical device;
visually observing the spiraling marking rotate along with the delivery wire; and
in response to rotating the delivery wire, releasing the medical device from its attachment to the delivery wire.

16. The method of claim 15, further comprising anchoring the medical device to the patient's body prior to rotating the delivery wire.

17. The method of claim 15, further comprising advancing the medical device out of the catheter wherein the rotatable connection is exposed from the catheter.

18. The method of claim 15 further comprising retracting the wire and the medical device prior to rotating the wire to reposition the medical device within the patient's body.

19. The method of claim 15, further comprising monitoring the position of the medical device relative to the delivery wire via fluoroscopy.

20. The method of claim 19 further comprising injecting a contrasting agent through the catheter prior to inserting the delivery device and medical device into the catheter.
